(19) United States
(12) Patent Application Publication
(10) Pub. No.: US 2012/0136015 A1
(43) Pub. Date: May 31, 2012

(54) PROCESS FOR PREPARATION OF ENDOTHELIAL RECEPTOR ANTAGONIST (BOSENTAN)

(75) Inventors: Shreerang Joshi, Thane (IN); Rashid Khan, Thane (IN); Deven Bendre, Thane (IN); Dadasaheb Salunkhe, Thane (IN); Sanket Gudekar, Thane (IN)

(73) Assignee: SANDOZ AG, Basel (CH)

(21) Appl. No.: 13/260,737

(22) PCT Filed: Apr. 9, 2010
(86) PCT No.: PCT/EP2010/054720

§ 371 Date: Feb. 16, 2012

(30) Foreign Application Priority Data
Apr. 13, 2009 (IN) 969/MUM/2009
Apr. 13, 2009 (IN) 971/MUM/2009

(51) Int. Cl. A61K 31/506; A61P 9/12; C07D 403/04

(52) U.S. Cl. 514/269; 544/296

(57) ABSTRACT

The present invention relates to processes for the preparation of an endothelial receptor antagonist. The present invention particularly relates to synthesis of 4-tert-butyl-N-[6-(2-hydroxyethoxy)-5-(2-methoxyphenoxy)-2-(2-pyrimidinyl)-4-pyrimidinyl]benzene sulfonamide (bosentan).

### FIELD OF THE INVENTION

The present invention relates to a process for the preparation of an endothelial receptor antagonist. More specifically it relates to the synthesis of 4-tert-butyl-N-[6-(2-hydroxyethoxy)-5-(2-methoxyphenoxy)-2-(2-pyrimidinyl)-4-pyrimidinyl]benzene sulfonamide (bosentan).

### BACKGROUND OF THE INVENTION

Bosentan represented by structural formula I is marketed under the brand name Tracleer® and is indicated for the treatment of pulmonary arterial hypertension (WHO Group I) in patients with WHO Class III of IV symptoms, to improve exercise ability and decrease the rate of clinical worsening.

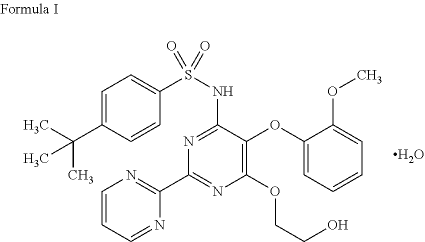

Bosentan was first disclosed in U.S. Pat. No. 5,292,740. The synthetic process disclosed for the preparation of Bosentan in this patent illustrates the conversion of 4,6-Dichloro-5-(2-methoxyphenoxy)-2,2′-bipyrimidine to 4-tert-butyl-N-[6-chloro-5-(2-methoxyphenoxy)-4-pyrimidinyl]benzenesulfonamide involving the use of very high temperatures and high boiling solvents such as DMSO.

The last step discloses the use of ethylene glycol and sodium metal for the conversion of 4-tert-butyl-N-[6-chloro-5-(2-methoxyphenoxy)-4-pyrimidinyl]benzene sulfonamide to Bosentan at a temperature of about 95° C. The disadvantage of this process is the formation of bis-sulfonamides (Formula II) in which two molecules of pyrimidine monohalide are coupled with one molecule of ethylene glycol. The removal of this bis-sulfonamide compound requires costly and laborious separation steps to obtain a pharmaceutically suitable pure Bosentan. In addition, the handling of sodium metal in two different steps on an industrial scale is hazardous.

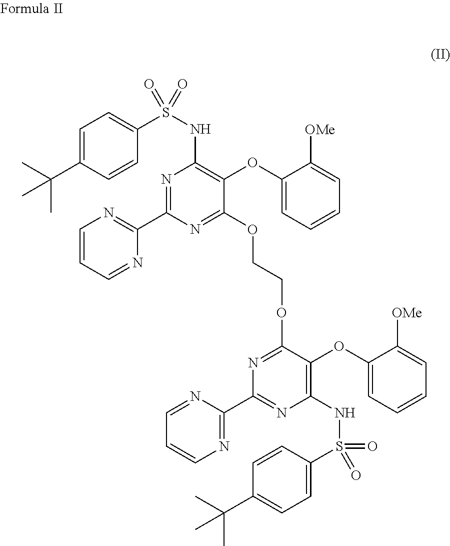

U.S. Pat. No. 6,136,971 disclose a process for the preparation of Bosentan with high purity. The patent discloses the use of mono-protected ethylene glycol and thus solves the problem of dimer formation. Tert-butyl group is used to protect one hydroxyl group of ethylene glycol as ether. The protecting group is then removed using formic acid to produce a formyloxy protected ethylene glycol sulfonamide derivative. Treatment of this compound with a base, preferably sodium hydroxide, then produces an ethylene glycol sulfonamide derivative containing a free hydroxyl group. This process involves many steps involving protection and deprotection of ethylene glycol as the tert-butyl ether and thus has limited commercial applications. Consequently, the process is not cost effective for commercial manufacture.

WO2009/004374 discloses a process for the synthesis of bosentan from 4-tert-butyl-N-[6-chloro-5-(2-methoxyphenoxy)-4-pyrimidinyl]benzenesulfonamide using ethylene glycol and hydroxide ions.

U.S. Pat. No. 6,121,447 discloses a process of preparation of bosentan with 2 cyanopyrimidine as the starting material. However, the steps involved in the said prior art are multiple thereby making the process expensive and laborious.

WO2009112954 discloses process of preparation of substantially pure ethylene glycol sulfonamide compounds such as bosentan using monoprotected ethylene glycol.

WO2009095933 discloses processes of preparation of bosentan from diethyl 2-(2-methoxyphenoxy)malonate and p-tert-butyl-N-[6-halo-5-(2-methoxyphenoxy)[2,2′-bipyrimidin]-4-yl]benzene sulfonamide as starting material.

A process for the preparation of 4-tert-butyl-N-[6-chloro-5-(2-methoxyphenoxy)-4-pyrimidinyl]benzenesulfonamide and bosentan therefrom, which is more direct, gives quantitative yield, is environmental friendly and applicable to industrial scale production, is desirable.

### SUMMARY OF INVENTION

Accordingly in a first aspect of the present invention there is provided an improved process for the preparation of bosentan comprising steps of:
a. preparation of (2-methoxyphenoxy)-2,2′-bipyrimidine (compound 1) by reacting 2-cyanopyrimidine with dimethyl 2-(2-methoxy phenoxy)malonate in presence of methanol, sodium methoxide and ammonium chloride at 25° C. to 30° C. without isolation of the intermediate, pyrimidine-2-carboxamidine hydrochloride;
b. reacting (2-methoxyphenoxy)-2,2′-bipyrimidine (compound 1) with phosphorus oxychloride to yield 4,6-Dichloro-5-(2-methoxyphenoxy)-2,2′-bipyrimidine (compound 2);
c. refluxing 4,6-Dichloro-5-(2-methoxyphenoxy)-2,2′-bipyrimidine (compound 2) with 4-tert-butylbenzene sulfonamide (compound 3) in presence of base and solvent to give 4-tert-butyl-N-[6-chloro-5-(2-methoxyphenoxy)-4-pyrimidinyl]benzenesulfonamide (compound 4);
d. reacting 4-tert-butyl-N-[6-chloro-5-(2-methoxyphenoxy)-4-pyrimidinyl]benzenesulfonamide (compound 4) with alkali metal amides or alkali metal hydrides and ethylene glycol to give crude bosentan;
e. isolating the crude bosentan;
f. purifying crude bosentan to obtain pure bosentan

According to yet another aspect of the present invention there is provided a process for the preparation of bosentan comprising the steps of:
a. preparation of (2-methoxyphenoxy)-2,2′-bipyrimidine (compound 1) by reacting 2-cyanopyrimidine with dimethyl 2-(2-methoxy phenoxy)malonate in presence of methanol, sodium methoxide and ammonium chloride at 25° C. to 30° C. without isolation of the intermediate, pyrimidine-2-carboxamidine hydrochloride
b. reacting (2-methoxyphenoxy)-2,2′-bipyrimidine (compound 1) with phosphorus oxychloride to yield 4,6-Dichloro-5-(2-methoxyphenoxy)-2,2′-bipyrimidine (compound 2)
c. refluxing 4,6-Dichloro-5-(2-methoxyphenoxy)-2,2′-bipyrimidine with 4-tert-butylbenzene sulfonamide (compound 3) in presence of bases and solvent to give 4-tert-butyl-N-[6-chloro-5-(2-methoxyphenoxy)-4-pyrimidinyl]benzenesulfonamide (compound 4);
d. reacting 4-tert-butyl-N-[6-chloro-5-(2-methoxyphenoxy)-4-pyrimidinyl]benzene sulfonamide (compound 4) with glycolaldehyde diethylacetal in presence of a base to form 4-tert-Butyl-N-[6-(2,2-diethoxy-ethoxy)-5-(2-methoxyphenoxy)-[2,2′]bipyrimidin-4-yl]-benzene sulphonamide in situ (compound 4A);
e. reacting 4-tert-butyl-N-[6-(2,2-diethoxy-ethoxy)-5-(2-methoxyphenoxy)-[2,2′]bipyrimidin-4-yl]-benzene sulphonamide (compound 4A) with aqueous acid to give 4-tert-Butyl-N-[5-(2-methoxyphenoxy)-6-(2-oxo-ethoxy)-[2,2′]bipyrimidin-4-yl]-benzene sulphonamide (compound 5);
f. reacting 4-tert-Butyl-N-[5-(2-methoxyphenoxy)-6-(2-oxo-ethoxy)-[2,2′]bipyrimidin-4-yl]-benzene sulphonamide (compound 5) with a reducing agent in a solvent to give crude bosentan;
g. isolating the crude bosentan;
h. purifying crude bosentan to obtain pure bosentan

It is another aspect of the present invention to provide a process for the preparation of bosentan free of Dimeric impurity

A further aspect of the present invention to provide a process for the preparation of Bosentan with HPLC purity greater than 99%.

Yet another aspect of the present invention relates to a pharmaceutical composition of Bosentan prepared according to the process of the present invention.

A further aspect of the present invention relates to a pharmaceutical composition comprising bosentan.

### DETAILED DESCRIPTION OF THE INVENTION

The subject of the present invention has now been described in terms of preferred embodiments.

In detail the present invention relates to a less expensive and less laborious processes for preparation of bosentan. However, these are to be construed as illustrative and non-limiting.

The inventors of the present invention have surprisingly found that (2-methoxy phenoxy)-2,2′-bipyrimidine can be prepared without isolating the intermediate pyrimidine-2-carboxamidine hydrochloride. In other words, 2-cyanopyrimidine can be converted to (2-methoxyphenoxy)-2,2′-bipyrimidine (compound 1) in a single step. This is affected by reacting 2-cyanopyrimidine with dimethyl 2-(2-methoxy phenoxy)malonate in presence of methanol, sodium methoxide and ammonium chloride. The reaction more preferably is carried at 25° C. to 30° C. The resulting (2-methoxyphenoxy)-2,2′-bipyrimidine can be used for the present invention.

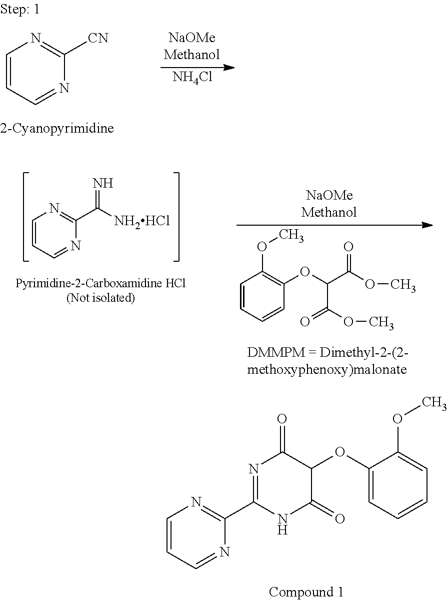

According to a preferred embodiment of the present invention the process of preparation of bosentan comprises reacting (2-methoxyphenoxy)-2,2′-bipyrimidine (compound 1) with phosphorus oxychloride to yield 4,6-Dichloro-5-(2-methoxyphenoxy)-2,2′-bipyrimidine (compound 2). This intermediate is refluxed with 4-tert-butylbenzene sulfonamide (compound 3) in presence of bases such as alkali metal hydroxides or carbonates and a solvent to give 4-tert-butyl-N-[6-chloro-5-(2-methoxyphenoxy)-4-pyrimidinyl]benzene sulfonamide (compound 4). The resultant is reacted with ethylene glycol in presence of alkali metal amides or alkali metal hydrides to give crude bosentan. The product obtained is purified with an organic solvent such as methanol and isopropyl acetate. The embodiment is represented hereinafter as scheme I.

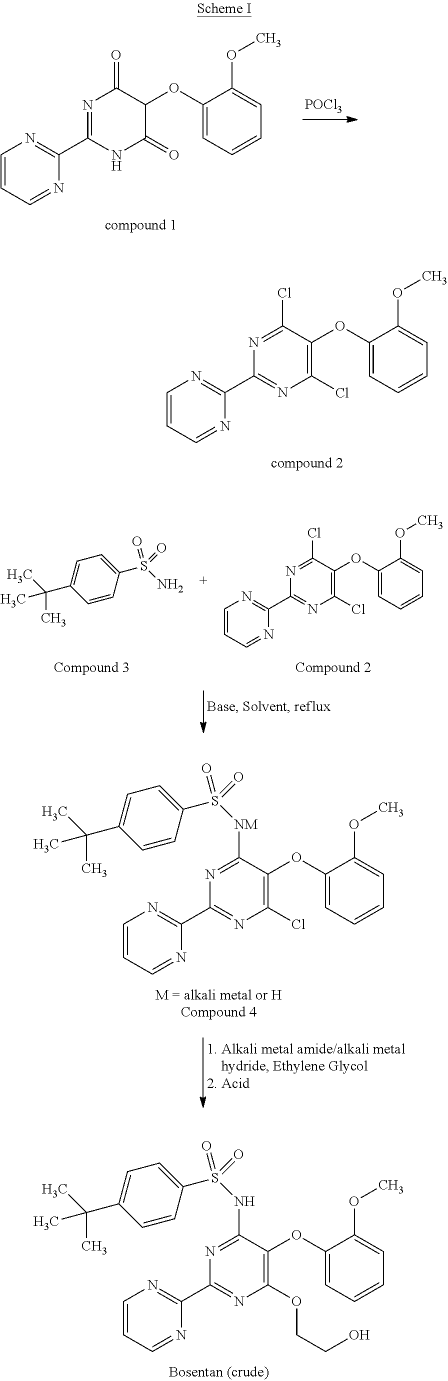

In an embodiment of the present invention the useful solvents include but not limited to acetone and toluene.

The base of the embodiment of the present invention include alkali metal hydroxide/carbonate include but are not limited to potassium hydroxide, potassium carbonate, sodium hydroxide, sodium carbonate, lithium hydroxide, lithium hydroxide monohydrate, or lithium carbonate.

The reaction temperature is the range of 40-120° C. In an embodiment of the present invention the reaction temperature is in the range of 50-110° C.

In an embodiment of the present invention the reaction mixture is refluxed for 6 to 7 hrs.

In an embodiment of the present invention, 4,6-Dichloro-5-(2-methoxyphenoxy)-2,2′-bipyrimidine (compound 2) is refluxed with 4-tert-butylbenzene sulfonamide (compound 3) in the presence of alkali metal hydroxide in acetone for a period of 4-8 hours.

In another embodiment of the present invention, 4,6-Dichloro-5-(2-methoxyphenoxy)-2,2′-bipyrimidine (compound 2) is refluxed with 4-tert-butylbenzene sulfonamide (compound 3) in the presence of alkali metal carbonate in toluene for a period of 5-8 hours

The alkali metal amide or alkali metal hydride of the present invention include but are not limited to sodium amide, sodium hydride, potassium hydride, lithium amide

In an embodiment of the present invention the reaction mixture of 4-tert-butyl-N-[6-chloro-5-(2-methoxyphenoxy)-4-pyrimidinyl]benzenesulfonamide (compound 4) and alkali metal amides or alkali metal hydrides and ethylene glycol is heated for 8 to 11 hrs.

In an embodiment of the present invention the ratio of ethylene glycol to 4-tert-butyl-N-[6-chloro-5-(2-methoxyphenoxy)-4-pyrimidinyl]benzenesulfonamide (compound 4) was in the range of 8-15 volumes.

In an embodiment of the present invention the ratio of sodium amide to 4-tert-butyl-N-[6-chloro-5-(2-methoxyphenoxy)-4-pyrimidinyl]benzenesulfonamide (compound 4) was in the range of 3-4 equivalents.

Also after the isolation of crude bosentan ethylene glycol which is used may be recovered from mother liquor and be reused. This further reduces the effluent load due to recovery and reuse of ethylene glycol.

According to preferred embodiment of the present invention the process of preparation of bosentan is comprises reacting (2-methoxyphenoxy)-2,2′-bipyrimidine (compound 1) with phosphorus oxychloride to yield 4,6-Dichloro-5-(2-methoxyphenoxy)-2,2′-bipyrimidine (compound 2). Refluxing the resultant intermediate with 4-tert-butylbenzene sulfonamide (compound 3) in presence of bases such as alkali metal hydroxides or carbonates and a solvent to give 4-tert-butyl-N-[6-chloro-5-(2-methoxyphenoxy)-4-pyrimidinyl]benzenesulfonamide (compound 4). Reacting the resultant with glycolaldehyde diethylacetal in presence of a base such as alkali metal hydride or alkali metal amide or alkali metal hydroxide to form 4-tert-Butyl-N-[6-(2,2-diethoxy-ethoxy)-5-(2-methoxyphenoxy)-[2,2′]bipyrimidin-4-yl]-benzene sulphonamide in situ (compound 4A); the intermediate is treated with aqueous acid to give 4-tert-Butyl-N-[5-(2-methoxyphenoxy)-6-(2-oxo-ethoxy)-[2,2′]bipyrimidin-4-yl]-benzene sulphonamide (compound 5). This is further reacted with reducing agent in a solvent to give crude bosentan. The product obtained is further treated with an organic solvent to obtain pure bosentan. The embodiment is represented hereinafter as Scheme II.

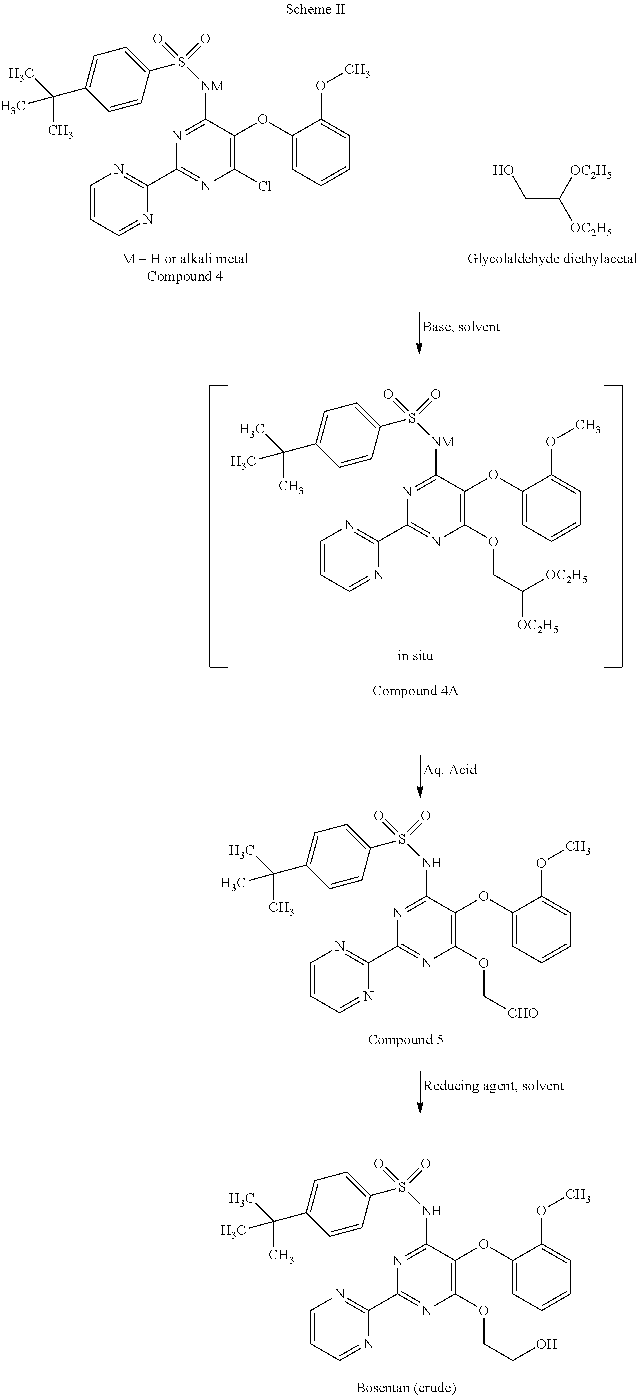

In an embodiment of the present invention reaction of 4-tert-butyl-N-[6-chloro-5-(2-methoxyphenoxy)-4-pyrimidinyl]benzene sulfonamide (compound 4) with glycolaldehyde dialkylacetal is in presence of base to give 4-tert-Butyl-N-[6-(2,2-diethoxy-ethoxy)-5-(2-methoxyphenoxy)-[2,2′]bipyrimidin-4-yl]benzene sulphonamide or its alkali metal salt (compound 4A)

The solvent that may be useful includes but is not limited to N,N-dimethyl formamide or dimethyl sulphoxide.

The bases of the preferred embodiment of the present invention includes but are not limited to sodium hydride, sodium amide, lithium amide, potassium hydroxide, lithium hydroxide, sodium hydroxide.

The glycolaldehyde dialkylacetal used in an embodiment of the present invention includes, but is not limited to glycolaldehyde dialkylacetal with alkyl corresponding to C1 to C4 carbon chain

The reaction temperature is the range of 10-70° C. In an embodiment of the present invention the reaction temperature is in the range of 30-65° C.

In an embodiment of the present invention the reaction mixture is stirred for 3 to 8 hrs, more preferably 3-5 hrs.

In the present invention, step (e) 4-tert-Butyl-N-[6-(2,2-diethoxy-ethoxy)-5-(2-methoxyphenoxy)-[2,2′]bipyrimidin-4-yl]benzene sulphonamide (compound 4A) is reacted with aq. acid to give 4-tert-Butyl-N-[5-(2-methoxyphenoxy)-6-(2-oxo-ethoxy)-2,2′]bipyrimidin-4-yl]-benzene sulphonamide (compound 5).

The reaction temperature of in final step is in the range of 20-40° C. More preferably the temperature is in the range of 25-35° C.

In an embodiment of the present invention the reaction mixture of 4-tert-Butyl-N45-(2-methoxyphenoxy)-6-(2-oxo-ethoxy)-[2,2′]bipyrimidin-4-yl]-benzene sulphonamide (compound 5) with a reducing agent in a solvent is stirred for 3 to 10 hrs.

In an embodiment of the present invention 4-tert-Butyl-N-[5-(2-methoxyphenoxy)-6-(2-oxo-ethoxy)-[2,2′]bipyrimidin-4-yl]-benzene sulphonamide (compound 5) is reacted with a reducing agent to give crude bosentan.

The reducing agent includes any metal hydride but is not limited to sodium borohydride, lithium aluminum hydride, sodium bis(2-methoxyethoxy)aluminum dihydride (Vitride).

The reaction time is in the range of 1-10 hrs. More specifically the reaction time is 4 hrs.

The process according to the invention as herein described yields bosentan with a high yield of around 70-80% and with >99% HPLC purity

The process according to the invention is free of dimeric impurity of formula II.

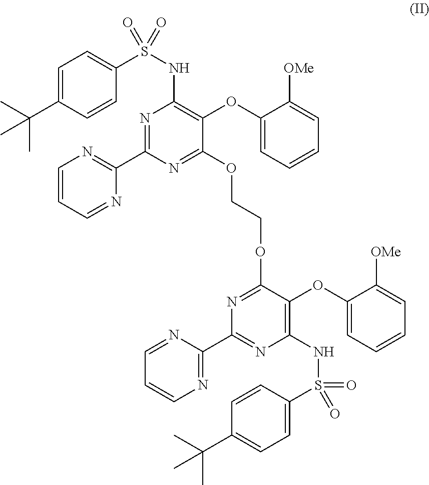

In another embodiment of the present invention, bosentan according to any one of the preceding aspects and embodiments, in the manufacture of a composition for the treatment or prevention of an endothelin-receptor mediated disorder.

Further embodiment of the present invention provides a pharmaceutical composition comprising bosentan according to any one of the preceding aspects and embodiments, and at least one pharmaceutically acceptable excipient.

Pharmaceutical formulations of the present invention contain bosentan and a pharmaceutically acceptable excipient. Pharmaceutical formulations of the present invention include but are not limited to tablets, powders, capsules, suppositories, sachets, troches and lozenges, as well as liquid syrups, suspensions and elixirs. Examples of such excipients are diluents, binders, anti-caking agents, solubilizers, disintegrants, fillers, lubricants, flavorants, stabilizers, colorants, dyes, anti-oxidants, anti-adherents, preservatives, glidants and carrier materials. A combination of excipients may also be used. Such excipients are known to those skilled in the art, and thus, only a limited number will be specifically referenced.

Binders which could be used include, but are not limited to, starches, e.g., potato starch, wheat starch, corn starch, pre-gelatinized starch; gums, such as gum tragacanth, acacia gum and gelatin; and polyvinyl pyrrolidone,

Fillers which could be used include, but are not limited to, microcrystalline cellulose [Avicel PH-101, Avicel PH-301, Avicel PH-102 SCG, Avicel HFE-102, Avicel PH-200 Avicel PH-302], starch, pre-gelatinized starch, modified starch, dibasic calcium phosphate dihydrate, calcium sulfate trihydrate, calcium sulfate dihydrate, calcium carbonate, dextrose, sucrose, lactose, mannitol and sorbitol.

Preferred diluents include, but are not limited to, dextrose, sorbitol, sucrose, lactose, mannitol, gelatin, starch, methyl cellulose, ethyl cellulose, propyl cellulose, hydroxymethyl cellulose, hydroxyethyl cellulose, hydroxypropyl cellulose, hydroxypropyl methyl cellulose, silica, polyvinyl alcohol, polyvinylpyrrolidone, cyclodextrins.

Disintegrants which could be used include but are not limited to natural starches, such as maize starch, potato starch and the like, directly compressible starches, e.g., Sta-rx® 1500; modified starches, e.g., carboxymethyl starches and sodium starch glycolate, available as Primojel®, Explotab®, Explosol®; and starch derivatives, such as amylase. Cross-linked polyvinylpyrrolidones, e.g., crospovidones, such as Polyplasdone® XL and Kollidon® CL. Alginic acid and sodium alginate. Methacrylic acid-divinylbenzene co-polymer salts, Cross-linked sodium carboxymethylcellulose, available as, e.g., Ac-di-sol®, Primellose®, Pharmacel® XL, Explocel® and Nymcel® ZSX. Additional disintegrants also include hydroxypropyl cellulose, hydroxypropylmethyl cellulose, croscarmellose sodium, sodium starch glycolate, polacrillin potassium, polyacrylates, such as Carbopol®, magnesium aluminium silicate and bentonite.

Lubricants include but are not limited to stearate salts of metals e.g. magnesium stearate, Sodium stearyl fumarate, hydrogenated vegetable oil Type I and II, Glyceryl dibehanate, zinc stearate.

Pharmaceutical compositions comprising bosentan according to the invention in the manufacture of a medicament for the treatment or prevention of an endothelin-receptor mediated disorder is a preferred embodiment of the present invention.

The pharmaceutical compositions encompass any composition comprising bosentan of the present invention manufactured with pharmaceutically acceptable excipients.

While this invention has been described in terms of specific embodiments, it should be understood that presented by way of illustration only and that the invention is not necessarily limited thereto. Modifications and variations within the spirit and scope of the claims that follow will be readily apparent from this disclosure, as those skilled in the art will appreciate.

### EXAMPLES

### Example 1

### 2-Cyanopyrimidine to 5-(2-methoxyphenyl)-2-(pyrimidin-2-yl)pyrimidin-4,6-(1H, 5H)-dione (compound 1)

900 ml of methanol and 100.0 gm of 2-cyanopyrimidine were charged at 25-30° C. and stirred for 5 minutes. 5.14 gm of sodium methoxide in 50.0 ml of methanol at 25-30° C. was charged and stirred for 3.0 hrs. Reaction progress was monitored by HPLC. 56.0 gm of ammonium chloride was charged and stirred at 25-30° C. for 3.0 hrs. Prepared a stock solution of 221.0 gm of sodium methoxide in 800.0 ml of methanol (Weight-781.0 gm). Added 599.0 gm of sodium methoxide to the reaction mass at 20-25° C. Stirred at 20-25° C. for 1.0 hr. Cooled the reaction mass to 20° C. Prepared a stock solution of 338.50 gm of DMMPM in 1.60 lit of methanol (Weight-1.5475 kg). Added 1.216 kg of the prepared stock solution of DMMPM to the reaction mass at 20-25° C. Stirred at 20-25° C. for 7.0 hrs. Remaining stock solution (182.0 gm) of sodium methoxide in methanol from the previously prepared stock solution was added to the reaction mass at 20-25° C. and stirred at 20-25° C. for 30.0 mins. The reaction mass was cooled to 20° C. Added remaining stock solution from the previously prepared stock solution, (331.8 gm) of DMMPM to the reaction mass at 20-25° C. Stirred at 20-25° C. for 5.0 hrs. Reaction progress monitored by HPLC. Distilled out solvent under vacuum completely at 45-50° C. Stripped out solvent under vacuum completely at 45-50° C. with 500.0 ml water twice. Added 1.50 lit of water slowly in the reaction mass at 25-30° C. and stirred for 1.0 hr. Added conc. HCl slowly at 25-30° C. till pH 3.5 to 4.0. (Required about 177.0 ml of conc. HCl). Stirred the reaction mass at 25-30° C. for 8.0 hrs. Cooled the reaction mass to 5-10° C. and stirred for 2.0 hrs. Filtered the reaction mass and washed the wet cake five times with 100.0 ml of water. Unloaded the wet material. Weight of wet compound 1: 238.90 gm. Dried the material under vacuum at 55-60° C. for 8.0 hrs. Weight of compound 1: 211.20 gm. Charged 1.0 lit of toluene to a 3 lit. RB assembly with Dean Stark apparatus at 25-30° C. Charged compound 1 and refluxed the reaction mass by using Dean Stark apparatus and removed water completely till the temperature attains between 105-110° C. After removal of water reflux the reaction mass for 1.0 hr. Cooled the reaction mass gradually to 25-30° C. and stirred for 1.0 hr. Filtered the reaction mass and washed the wet material with 100.0 ml of toluene. Unloaded the wet material. Weight of wet compound 1: 207.20 gm. Dried the material under vacuum at 55-60° C. for 5.0 hrs. Weight of compound 1: 185.60 gm (% Yield: 62%, HPLC Purity: >98%)

### Example 2

### 5-(2-methoxyphenyl)-2-(pyrimidin-2-yl)pyrimidin-4,6-(1H,5H)-dione (compound 1) to 4,6-dichloro-5-(2-methoxyphenoxy)-2,2′-bipyrimidine (compound 2)

Charged 343.70 gm of phosphorous oxychloride followed by 175.0 gm of compound 1. Raised the temperature of reaction mass to reflux. Stirred the reaction mass at reflux for 4.0 hr. Reaction is monitored by HPLC. Cooled the reaction mass gradually to 40-50° C. Quenched the reaction mass slowly into 2.625 lit of water at 5-10° C. Stirred the reaction mass at 5-10° C. for 2.0 hrs. Filtered and washed the wet material thrice with 175.0 ml of water. Unloaded the wet material. Weight wet of Compound 2: 255 gm. Dried the wet material under vacuum at 55-60° C. for 8.0 hrs. Weight of dried compound 2: 182 gm (% Yield: 93%; HPLC Purity: >98%))

### Example 3

### Preparation of 4-tert-butyl-N-[6-chloro-5-(2-methoxyphenoxy)-4-pyrimidinyl]benzene sulfonamide potassium salt using Acetone as a solvent and potassium hydroxide as a base

Acetone (45.0 ml), Potassium hydroxide (1.13 gm) and 4-tert-butylbenzenesulphonamide (1.65 gm) were added at 30° C. and stirred for 5 minutes. 4,6-dichloro-5-(2-methoxyphenoxy)-2,2′-bipyrimidine (3.0 gm) was added at 30° C. and the temperature of the reaction mass was raised to reflux. The reaction mass was stirred at reflux for 6.5 hrs. The reaction mass was cooled gradually to room temperature. Acetone was distilled out from the reaction mass under vacuum below 40° C. Water (30.0 ml) was added to the reaction mass at room temperature and the resulting mass was stirred for 3.0 hrs. The precipitated solid was filtered, washed with water (2×3.0 ml) and dried under vacuum at 55-60° C. for 6.0 hrs to obtain 4.04 gms of 4-tert-butyl-N-[6-chloro-5-(2-methoxyphenoxy)-4-pyrimidinyl]benzene sulfonamide potassium salt. (% Yield: 89%)

MS of solid: 564.1 (M+H), 548.1, 514.2, 434.1, 370.0, 352.1, 340.0, 324.1, 249.2, 237.1, 197.6, 182.9

### Example 4

### Preparation of 4-tert-butyl-N-[6-chloro-5-(2-methoxyphenoxy)-4-pyrimidinyl]benzene sulfonamide potassium salt using Toluene as a solvent and potassium carbonate as a base

Toluene (150.0 ml), Potassium carbonate (11.87 gm) and 4-tert-butylbenzenesulphonamide (6.72 gm) were added at 30° C. and stirred for 5 minutes. 4,6-dichloro-5-(2-methoxyphenoxy)-2,2′-bipyrimidine (10.0 gms) was added at 30° C. and the temperature of the reaction mass was raised to reflux. The reaction mass was stirred at reflux for 7.0 hrs. The reaction mass was cooled gradually to room temperature. Stirred the reaction mass at room temperature for 1 hr. The precipitated solid was filtered, washed with toluene (2×10.0 ml) and dried under vacuum at 55-60° C. for 5.0 hrs to obtain 13.16 gms of 4-tert-butyl-N-[6-chloro-5-(2-methoxyphenoxy)-4-pyrimidinyl]benzenesulfonamide potassium salt. (% Yield: 87%)

MS of solid: 564.1 (M+H), 547.9, 513.9, 434.1, 352.1, 237.1, 198.0, 183.0

### Example 5

### Preparation of Bosentan from Compound 4 and Ethylene Glycol Using Sodium Amide as a Base

Ethylene Glycol (15.0 ml) and Sodium amide (1.13 gm) were added at 30° C. and stirred for 5 minutes. The temperature of the reaction mass was raised to 50° C. and stirred at 50° C. for 1.0 hr. The reaction mass was cooled gradually to room temperature and 4-tert-butyl-N-[6-chloro-5-(2-methoxyphenoxy)-4-pyrimidinyl]benzene sulfonamide (3.0 gm) was added. Ethylene Glycol (9.0 ml) was added to the reaction mass and the temperature of the reaction mass was raised to 70° C. The reaction mass was stirred at 70-75° C. for 10.0 hrs. The reaction mass was cooled to room temperature. Water (30.0 ml) was added to the reaction mass at room temperature and the resulting mass was stirred for 15 minutes. Conc. HCl (5.0 ml) was added to the reaction mass and stirred for 3.0 hrs. The precipitated solid was filtered, washed with water (3×3.0 ml) and dried under vacuum at 55-60° C. for 10.0 hrs to obtain 2.34 gms of Bosentan. (% Yield: 72%)

MS of solid: 552.2 (M+H), 508.3, 400.4, 311.2, 280.2, 202.1

### Example 6

### Preparation of Bosentan from Compound 4 and Ethylene Glycol Using Sodium Amide as a Base

Sodium amide (14.85 gm) were added at 5-10° C. to Ethylene Glycol (375.0 ml) and stirred for 5 minutes. The temperature of the reaction mass was raised to 75° C. and stirred at 75-80° C. for 0.5 hr. The reaction mass was cooled gradually to 25-30° C. and 4-tert-butyl-N-[6-chloro-5-(2-methoxyphenoxy)-4-pyrimidinyl]benzene sulfonamide (25.0 gm) was added. The temperature of the reaction mass was raised to 75-80° C. The reaction mass was stirred at 75-80° C. for 20.0 hrs. The reaction mass was cooled to room temperature and then 5-10° C. Stirred for 30 minutes. Filtered and washed with Ethylene Glycol (12.5 ml) to get the wet cake of sodium salt of Bosentan (wet cake wt. 37.38 gm). Charged wet cake in water (250.0 ml) and Methanol (25.0 ml) and stirred for 30 minutes. Charged Conc. HCl slowly till pH 1-2 (required qty 22.0 ml). stirred at room temp. for 5.0 hrs. The precipitated solid was filtered, washed with water (2×25.0 ml) and dried under vacuum at 55-60° C. for 10.0 hrs to obtain 23.65 gms of Bosentan. (% Yield: 87.4%)

### Example 7

### Preparation of 4,6-dichloro-5-(2-methoxyphenoxy)-2,2′-bipyrimidine (compound 2) to 4-tert-butyl-N-[6-chloro-5-(2-methoxyphenoxy)-4-pyrimidinyl]benzene sulfonamide (compound 4)

Charged 2.580 lit. of acetone, 94.50 gm of 4-tert-butyl benzene sulphonamide at 25-30° C. and stirred for 5.0 min. Charged 65.0 gm of potassium hydroxide and stirred at 25-30° C. for 15.0 min. Charged 172.0 gm of compound 2 at 25-30° C. Raised the temperature of reaction mass to reflux. Stirred the reaction mass at reflux for 7.0 hrs and monitored the reaction by HPLC for content of compound 2 for each hour. Cooled gradually to 40° C. Distilled out acetone completely under vacuum below 45° C. Added 172.0 ml of acetone and 1.720 lit of water to the reaction mass at 25-30° C. Stirred at 25-30° C. for 15.0 min. Prepared a 10% hydrochloric acid solution by adding 57.20 ml of conc.HCl in 200.0 ml of water. Added the above 10% hydrochloric acid solution slowly at 25-30° C. till pH is 1-2. Stirred at 25-30° C. for 5.0 hrs. Filtered and washed the wet cake twice with 172.0 ml of water. Wet weight of crude compound 4-405.0 g.

Charged 860.0 ml of acetonitrile to another 2 lit. RB-assembly at 25-30° C. Charged wet 415.0 gm crude bosentan at 25-30° C. Raise the temperature of reaction mass to reflux. Refluxed the reaction mass for 1.0 hr. Cooled the reaction mass gradually to 25-30° C. Stirred the reaction mass at 25-30° C. for 2.0 hr. Filtered the reaction mass and washed the wet cake with 172.0 ml of chilled acetonitrile. Unload the wet material. Weight of wet bosentan: 223.20 gm. Dried the material under vacuum at 55-60° C. for 8.0 hrs.

Weight of dried compound 4: 175.50 gm (% Yield: 68%; HPLC Purity: >98%)

### Example 8

### Preparation of 4-tert-Butyl-N-[6-(2,2-diethoxy-ethoxy)-5-(2-methoxyphenoxy)-[2,2′]bipyrimidin-4-yl]-benzene sulphonamide (compound 4A) using NaH and DMF

4-t-butyl-N-[6-chloro-5-(o-methoxyphenoxy)-4-pyrimidinyl]benzenesulfonamide, potassium salt (3.0 gm, 0.0053 mol) was dissolved in N,N-dimethyl formamide (10 ml) at 30° C. under stirring. Glycolaldehyde diethylacetal (2.13 gm; 0.01587 mol) was added, followed by sodium hydride (60% dispersion in mineral oil) (1.35 gm, 0.03375 mol). The reaction mass was stirred for 30-40 minutes after the addition of sodium hydride at 45-50° C. and at 30° C. for 3 hrs. Water (60 ml) was added to the reaction mass and acidified with Conc. HCl (1.0 ml). The resulting solution was stirred for 30 minutes at 30° C. and extracted with 2×25 ml of Ethyl Acetate.

Combined Ethyl Acetate layer was washed with water (40 ml), dried over anhydrous sodium sulphate and evaporated under vacuum at 55-60° C. to give the diacetal compound as an oil (Weight of oil=4.63 gm).

MS of oil: 624.2 [M+H], 578.2, 550.2, 508.2, 336.0, 310.9, 202.3

### Example 9

### Preparation of 4-tert-Butyl-N-[5-(2-methoxyphenoxy)-6-(2-oxo-ethoxy)-[2,2′]bipyrimidin-4-yl]-benzene sulphonamide (Compound 5)

Conc. HCl (18.5 ml) was added to the oil of 2 (4.0 gm) and stirred for 1 hr. Water (37 ml) was added and stirred for 6 hrs. The solid precipitated was filtered, washed with water (2×9 ml) and dried under vacuum at 50-55° C. for 5 hrs to obtain the 4-tert-Butyl-N-[5-(2-methoxyphenoxy)-6-(2-oxo-ethoxy)-[2,2′]bipyrimidin-4-yl]benzenesulphonamide (3) as a yellow solid (Weight of the solid=2.5 gm). (% Yield: 85.6%)

MS of solid: 550.6 [M+H], 336.1, 310.2, 279.2, 202.1

IR (KBr): 3205, 2960, 2925, 2854, 1741, 1619, 1580, 1558

### Example 10

### Preparation of Bosentan (Crude)

To the solution of 4-tert-Butyl-N-[5-(2-methoxyphenoxy)-6-(2-oxo-ethoxy)-[2,2′]bipyrimidin-4-yl]-benzene sulphonamide (2.0 gm, 0.00363 mol) in Methanol (20 ml) was added Sodium borohydride (0.28 gm, 0.00726 mol) slowly and portion wise (a brisk effervescence was observed). The reaction mass was stirred for 4 hrs. Water (200 ml) was added to the reaction mixture and methanol was distilled out from the resulting mixture under vacuum at 50° C. The aqueous residue was cooled to 10-20° C., acidified Conc. HCl and stirred for 30 minutes. The solid precipitated was filtered, washed with water and dried under vacuum at 50-55° C. for 8 hrs to obtain the bosentan as a solid (Weight of the solid=1.26 gm). (% Yield: 60.8%)

MS of solid: 552.2 [M+H], 508.2, 311.2, 280.2, 202.1

### Example 11

### Preparation of 4-tert-Butyl-N-[6-(2,2-diethoxy-ethoxy)-5-(2-methoxyphenoxy)-[2,2′]bipyrimidin-4-yl]-benzenesulphonamide (compound 4A) using KOH and DMSO

4-tert-butyl-N-[6-chloro-5-(o-methoxyphenoxy)-4-pyrimidinyl]benzenesulfonamide (6.0 gm, 0.0114 mol), KOH (2.26 gm, 0.040 mol) and glycolaldehyde diethylacetal (4.6 gm, 0.0342 mol) were added to dimethyl sulphoxide (60 ml) at 30° C. The reaction mixture was heated to 65° C. and maintained under stirring for 5 hrs. The reaction mixture was cooled to 30° C., water (120 ml) was added slowly and stirred for 2 hrs at 30° C. The suspension of the product was filtered and washed with water (4×30 ml).

Weight of the Wet solid=7.83 gm

### Example 12

### Preparation of 4-tert-Butyl-N-[5-(2-methoxyphenoxy)-6-(2-oxo-ethoxy)-[2,2′]bipyrimidin-4-yl]-benzene sulphonamide (Compound 5)

Conc. HCl (31.5 ml) was added to wet Compound 4A (7.83 gm) and stirred for 1 hr. Water (63 ml) was added and stirred further for 6 hrs. The precipitated solid was filtered, washed with water (3×10 ml) and dried under vacuum at 55-60° C. for 5 hrs to obtain 3.29 gm of the 4-tert-Butyl-N-[5-(2-methoxyphenoxy)-6-(2-oxo-ethoxy)-[2,2′]bipyrimidin-4-yl]benzene-sulphonamide (Compound 5) as a yellow solid. (% Yield: 52.5%)

### Example 13

### Preparation of 4-tert-Butyl-N-[5-(2-methoxyphenoxy)-6-(2-oxo-ethoxy)-[2,2′]bipyrimidin-4-yl]-benzene sulphonamide (Compound 5)

4-t-butyl-N-[6-chloro-5-(o-methoxyphenoxy)-4-pyrimidinyl]benzenesulfonamide (15.0 gm, 0.029 mol), glycolaldehyde diethylacetal (11.5 gm, 0.086 mol) and KOH (5.65 gm, 0.086 mol) were added to dimethyl sulphoxide (150 ml) at 30° C. and heated to 65° C. and further stirred for 5 hrs. The reaction mixture was cooled to 30° C., water (300 ml) was added slowly and the resulting mixture stirred for 3 hrs at 30° C. The suspension was filtered and washed with water (5×15 ml) to give the wet cake of 4-tert-Butyl-N-[6-(2,2-diethoxy-ethoxy)-5-(2-methoxyphenoxy)-[2,2′]bipyrimidin-4-yl]-benzenesulphonamide (Compound 4A).

Conc. HCl (30 ml) was added to the wet cake of (Compound 4A) and stirred for 1 hr. Water (60 ml) was added and stirred for 6 hrs. The solid precipitated was filtered, washed with water (5×15 ml) and dried under vacuum at 50-55° C. for 8 hrs to obtain 8.15 gm of the 4-tert-Butyl-N-[5-(2-methoxyphenoxy)-6-(2-oxo-ethoxy)-[2,2]bipyrimidin-4-yl]benzenesulphonamide (Compound 5) as a yellow solid. (% Yield: 52.0%)

MS of solid: 552.2 [M+H], 508.2, 400.3, 311.2, 280.2, 202.1

### Example 14

### Purification of Crude Bosentan

Bosentan (crude) at room temperature was charged in a RB assembly equipped with a reflux condenser and 12.0 ml Isopropyl Acetate at room temperature and 3.0 ml Methanol at room temperature was charged. Reaction mass was heated to 70-75° C. and stirred for 10 mins at same temperature. It was gradually cooled to room temperature and further cooled to 5-10° C. and maintained for 2 hrs at the same temperature. The solid was filtered and washed with 3.0 ml of chilled Isopropyl Acetate and dried at 50-55° C. for 3 hrs. (1^{st} purified solid). This was repeated twice and the material obtained was heated at 70-75° C. in a mixture of 19.8 ml methanol and 19.8 ml water. After attaining the temperature of 70-75° C. 14 ml of methanol was added. The solid dissolves slowly at this temperature. Heating was stopped and allowed to cool the reaction mass gradually to room temperature. Solid crystals are formed slowly while stirring at room temperature for 2 hrs. The solid was filtered and washed with 10.0 ml of distilled water and dried at 55-60° c. for 8 hrs

1-11. (canceled)

12. An improved process for the preparation of bosentan comprising the steps of:
a) preparation of (2-methoxyphenoxy)-2,2′-bipyrimidine (compound 1) by reacting 2-cyanopyrimidine with dimethyl 2-(2-methoxy phenoxy)malonate in presence of methanol, sodium methoxide and ammonium chloride at 25° C. to 30° C. without isolation of the intermediate, pyrimidine-2-carboxamidine hydrochloride;
b) reacting (2-methoxyphenoxy)-2,2′-bipyrimidine (compound 1) with phosphorus oxychloride to yield 4,6-Dichloro-5-(2-methoxyphenoxy)-2,2′-bipyrimidine (compound 2);
c) refluxing 4,6-Dichloro-5-(2-methoxyphenoxy)-2,2′-bipyrimidine (compound 2) with 4-tert-butylbenzene sulfonamide (compound 3) in presence of base and solvent to give 4-tert-butyl-N-[6-chloro-5-(2-methoxyphenoxy)-4-pyrimidinyl]benzenesulfonamide (compound 4);
d) reacting 4-tert-butyl-N-[6-chloro-5-(2-methoxyphenoxy)-4-pyrimidinyl]benzenesulfonamide (compound 4) with alkali metal amides or alkali metal hydrides and ethylene glycol to give crude bosentan;
e) isolating the crude bosentan; and
f) purifying crude bosentan to obtain pure bosentan.

13. An improved process for the preparation of bosentan comprising the steps of:
a) preparation of (2-methoxyphenoxy)-2,2′-bipyrimidine (compound 1) by reacting 2-cyanopyrimidine with dimethyl 2-(2-methoxy phenoxy)malonate in presence of methanol, sodium methoxide and ammonium chloride at 25° C. to 30° C. without isolation of the intermediate, pyrimidine-2-carboxamidine hydrochloride;
b) reacting (2-methoxyphenoxy)-2,2′-bipyrimidine (compound 1) with phosphorus oxychloride to yield 4,6-Dichloro-5-(2-methoxyphenoxy)-2,2′-bipyrimidine (compound 2);
c) refluxing 4,6-Dichloro-5-(2-methoxyphenoxy)-2,2′-bipyrimidine (compound 2) with 4-tert-butylbenzene sulfonamide (compound 3) in presence of bases and solvent to give 4-tert-butyl-N-[6-chloro-5-(2-methoxyphenoxy)-4-pyrimidinyl]benzenesulfonamide (compound 4);
d) reacting 4-tert-butyl-N-[6-chloro-5-(2-methoxyphenoxy)-4-pyrimidinyl]benzene sulfonamide (compound 4) with glycolaldehyde diethylacetal in presence of a base to form 4-tert-Butyl-N-[6-(2,2-diethoxy-ethoxy)-5-(2-methoxyphenoxy)-[2,2′]bipyrimidin-4-yl]-benzene sulphonamide in situ (compound 4A);
e) reacting 4-tert-butyl-N-[6-(2,2-diethoxy-ethoxy)-5-(2-methoxyphenoxy)-[2,2′]bipyrimidin-4-yl]-benzene sulphonamide (compound 4A) with aqueous acid to give 4-tert-Butyl-N-[5-(2-methoxyphenoxy)-6-(2-oxo-ethoxy)-[2,2′]bipyrimidin-4-yl]-benzene sulphonamide (compound 5);
f) reacting 4-tert-Butyl-N-[5-(2-methoxyphenoxy)-6-(2-oxo-ethoxy)-[2,2′]bipyrimidin-4-yl]-benzene sulphonamide (compound 5) with a reducing agent in a solvent to give crude bosentan;
g) isolating the crude bosentan; and
h) purifying crude bosentan to obtain pure bosentan.

14. The process for the preparation of bosentan according to claim 13 which is free of dimeric impurity of formula II.

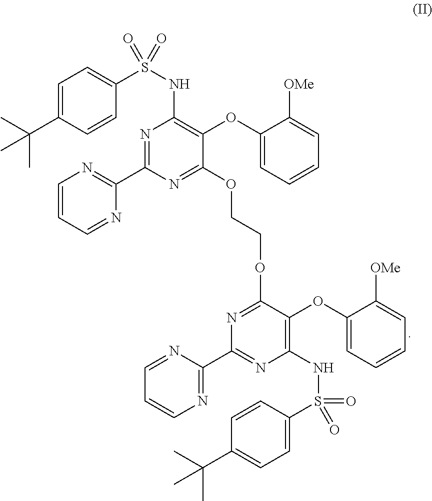

15. The process of preparation of bosentan according to claim 12, wherein the base is selected from the group of alkali metal hydroxides, alkali metal carbonates, alkali metal hydride or alkali metal amide.

16. The process of preparation of bosentan according to claim 13, wherein the base is selected from the group of alkali metal hydroxides, alkali metal carbonates, alkali metal hydride or alkali metal amide.

17. The process of preparation of bosentan according to claim 15, wherein the base is selected from potassium hydroxide, potassium carbonate, sodium hydroxide, sodium carbonate, lithium hydroxide, lithium hydroxide monohydrate, lithium carbonate, sodium hydride, sodium amide, lithium amide or potassium hydride.

18. The process of preparation of bosentan according to claim 16, wherein the base is selected from potassium hydroxide, potassium carbonate, sodium hydroxide, sodium carbonate, lithium hydroxide, lithium hydroxide monohydrate, lithium carbonate, sodium hydride, sodium amide, lithium amide or potassium hydride.

19. The process of preparation of bosentan according to claim 12, wherein the alkali metal amides and alkali metal hydride of step d is selected from sodium amide, sodium hydride, potassium hydride or lithium amide.

20. The process of preparation of bosentan according to claim 12, wherein the solvent is selected from acetone, toluene, N,N-dimethyl formamide, dimethyl sulphoxides, or acetonitrile.

21. The process of preparation of bosentan according to claim 13, wherein the solvent is selected from acetone, toluene, N,N-dimethyl formamide, dimethyl sulphoxides, or acetonitrile.

22. The process of preparation of bosentan according to claim 13, wherein the reducing agent is selected from sodium borohydride, lithium aluminium hydride, sodium bis (2 methoxyethoxy)aluminum dihydride.

23. The process of preparation of bosentan according to claim 12, wherein the purity of Bosentan is greater than 99% as measured by HPLC.

24. The process of preparation of bosentan according to claim 13, wherein the purity of Bosentan is greater than 99% as measured by HPLC.

25. The process of preparation of bosentan according to claim 14, wherein the purity of Bosentan is greater than 99% as measured by HPLC.

26. The process of preparation of bosentan according to claim 12, comprising less than about 0.1% of dimeric impurity.

27. The process of preparation of bosentan according to claim 13, comprising less than about 0.1% of dimeric impurity.

28. The process of preparation of bosentan according to claim 14, comprising less than about 0.1% of dimeric impurity.

29. Pharmaceutical compositions comprising 4-tert-butyl-N-[6-(2-hydroxyethoxy)-5-(2-methoxyphenoxy)-2-(2-pyrimidinyl)-4-pyrimidinyl]benzene sulfonamide, in accordance with claim 12, and a pharmaceutically acceptable carrier.

30. Pharmaceutical compositions comprising 4-tert-butyl-N-[6-(2-hydroxyethoxy)-5-(2-methoxyphenoxy)-2-(2-pyrimidinyl)-4-pyrimidinyl]benzene sulfonamide, in accordance with claim 13, and a pharmaceutically acceptable carrier.
